(19) United States
(12) Patent Application Publication
(10) Pub. No.: US 2006/0286148 A1
(43) Pub. Date: Dec. 21, 2006

(54) Method of forming implants

(75) Inventors: Beth Hollister, Hillsboro, NC (US); Robert Kaiser, Fuquay Varina, NC (US)

Correspondence Address: SHERIDAN ROSS PC, 1560 BROADWAY, SUITE 1200, DENVER, CO, 80202, US

(73) Assignee: PPD, Inc., Rockville, MD (US)

(21) Appl. No.: 11/415,505

(22) Filed: Apr. 27, 2006

Related U.S. Application Data
(60) Provisional application No. 60/684,804, filed on May 18, 2005.

(51) Int. Cl. A61L 33/00; B05D 3/00; A61K 38/18

(52) U.S. Cl. 424/427; 427/2.1; 264/299; 514/12

(57) ABSTRACT

The present invention is a method for producing an implant for a corneal pocket assay by introducing a solution into a perforated plate or member, allowing the solution to form pellets, and removing the pellets from the perforations. The present invention also includes a method for performing a corneal pocket assay for testing a putative pharmaceutically active agent into laboratory animals by placing a pellet into the corneal pocket of the laboratory animal, introducing a pharmaceutically active agent into the laboratory animal, and evaluating the pharmaceutical activity of the agent. The present invention also includes pellets and implants for use in a corneal pocket assay produced by the methods of the present invention. The present invention includes a plate for forming an implant for a corneal pocket assay. The present invention also discloses a method of making pills and pills produced thereby.

### CROSS-REFERENCE TO RELATED APPLICATION

This application claims the benefit of priority under 35 U.S.C. § 119(e) from U.S. Provisional Application Ser. No. 60/684,804 filed May 18, 2005. The entire disclosure of U.S. Provisional Application Ser. No. 60/684,804 is incorporated herein by reference.

### FIELD OF THE INVENTION

The invention relates to methods of producing implants, methods for performing corneal pocket assays, and devices for forming implants. Such methods and devices are suitable for corneal pocket assays and are particularly suitable for corneal pocket anti-angiogenesis assays in laboratory animals for identifying and evaluating putative angiogenesis inhibitors.

### BACKGROUND

In the field of neovascular research, the testing of angiogenesis and antiangiogenic substances relies on the sensitivity of in vivo and in vitro assays. Some of the more important in vivo methods are the chorioallantoic membrane assay, monkey iris neovascularization model, disc angiogenesis assay, and various others using the cornea to assess blood vessel growth. The visibility, accessibility, and avascularity of the cornea are advantageous and facilitate the biomicroscopic grading of the neovascular response and the topical application of test drugs. Several corneal angiogenesis models in the rabbit have been described, including direct intrastromal injections of substances, chemical or thermal injury, and intrastromal tumor implantation. Recently developed micropocket assays containing either E. coli-derived endotoxin or the intrastromal implantation of sustained release pellets containing basic fibroblast growth factor (bFGF) and sucralfate in the rabbit eye have also induced a reproducible angiogenic response.

The sustained release bFGF assay is a preferred model among researchers for the reason that it gives a predictable, persistent, and aggressive neovascular response, which is dependent on direct stimulation of blood vessels rather than on indirect stimulation by the induction of inflammation. In this particular assay, the potent angiogenic growth factor, bFGF, is complexed with a stabilizing agent such as sucralfate, which acts to stabilize the growth factor and slow its release from the polymer. This assay may be performed on laboratory animals, including rabbits and mice. The basic procedure is to form a corneal pocket by incision into the cornea and place a pellet containing the requisite components, including an angiogenesis stimulator such as bFGF, a stabilizing compound such as sucralfate, and a binding polymer such as hydroxyethylmethacrylate into the corneal pocket. A putative angiogenesis inhibitor is then administered to the laboratory animal. Inhibition of angiogenesis is then evaluated.

Known methods for forming the pellets for micropocket assays include the nylon mesh method, as described in Kenyon et al. A Model of Angiogenesis in the Mouse Cornea, Investigative Ophthalmology and Visual Science, Vol. 37 No. 8, pp 1625-1632 (1996). Nylon mesh with approximate pore size of 0.4 mm by 0.4 mm is used. Suspensions of sterile saline solution containing the appropriate amount of each component or ingredient of the pellet are embedded between the fibers, resulting in a grid of squares. In one type of nylon mesh, the result is a 15×15 grid of squares. The solution in the mesh is then allowed to dry for an appropriate length of time. Once the solution has dried, the fibers of the mesh are pulled apart under a microscope, and pellets are selected with the aid of a dissecting microscope for implantation. Among the approximately 200 pellets produced, Kenyon et al. report that only 30 to 40 uniformly sized pellets of 0.4×0.4×0.2 mm are suitable for use.

The above-described nylon mesh method for forming pellets for implant and use in corneal pocket assays has several drawbacks. One limitation to the usefulness of the above method for forming pellets is the variability of the pellets formed in this manner. Pellets formed in this manner are prone to be irregularly formed and/or misshapen. There is significant pellet-to-pellet dimensional variation as well as variances in the volume of each pellet. Due to the variability inherent in the above method for forming pellets, the prior art describes a yield of only 30-40 uniformly sized pellets out of approximately 200 pellets produced. This pellet waste is disadvantageous for a number of reasons. Pellet waste increases costs. The components used to make the pellets are expensive. Pellet waste also adds significantly to technician time in forming the pellets, also increasing costs. Finally, irregularly formed/misshapen or pellets with a greater variance in size add to the standard error of experiments performed with these pellets. Increasing standard error in experiments leads to greater uncertainty in the data generated and causes interpretation of the data to be more difficult.

In light of the shortcomings of the methods of forming pellets taught in the art, there is a need for pellets for corneal pocket assays with reduced pellet-to-pellet variability. Another need exists for devices capable of forming such pellets and methods for forming such pellets. These and other needs are answered by the present invention.

### SUMMARY OF THE INVENTION

In a first embodiment, the present invention includes a method for producing an implant for a corneal pocket assay. This method includes introducing a curable solution into perforations in a rigid plate, allowing the solution in the perforations to cure to the pellets produced by the method are substantially uniform in dimension. In another embodiment, the method of the present invention also includes a method for producing an implant for a corneal pocket assay which includes the steps of introducing a solution into perforations in a perforated member, allowing the solution in the perforations to form pellets, and removing pellets from the perforated member by pushing individual pellets through individual perforations.

Preferably, at least about 70%, at least about 80%, at least about 90% and most preferably, at least about 95% of the pellets are suitable for use in the corneal pocket assay. A preferred material for the plate is metal, most preferably stainless steel. The plate is preferably about 0.2 mm in thickness and the perforations are preferably round in cross-section and are about 0.5 mm in diameter. Preferably, the perforations are separated by at least about 0.2 mm. In preferred embodiments, the plate contains 100 perforations in a 10×10 grid.

The curable solution preferably includes a growth factor. A preferred growth factor includes bFGF and/or VEGF. Preferably, the solution also includes a slow-release agent, preferably hydroxyethylmethacrylate. The solution preferably also further includes a stabilizing agent, preferably sucralfate. Accordingly, a most preferred solution according to the present invention includes sucralfate, hydroxyethylmethacrylate, and a growth factor.

As described above, the method of the present invention also includes a removal step, which in a preferred embodiment includes pushing individual pellets through individual perforations. In most preferred embodiments, the present invention includes a removal step which is accomplished by pushing individual pellets through individual perforations with a blunted needle.

In another embodiment, the present invention includes a method of performing a corneal pocket assay for testing a putative pharmaceutically active agent in laboratory animals. This method includes the steps of placing a pellet into a corneal pocket of each of at least two laboratory animals, wherein the pellets are substantially uniform in dimension; introducing the pharmaceutically active agent into at least one of the at least two laboratory animals; and evaluating the pharmaceutical activity of the agent. Preferably, the pellets are produced by the methods of the present invention. In a preferred embodiment, the putative pharmaceutically active agent comprises a putative anti-angiogenesis agent. Preferably, the pellet further includes a growth factor, preferably bFGF and/or VEGF. Preferred pellets also include a slow-release agent, preferably hydroxyethylmethacrylate, and a stabilizing agent, preferably sucralfate. Preferred pellets comprise sucralfate, hydroxyethylmethacrylate, and a growth factor. In a preferred embodiment, the laboratory animal is a mouse.

The present invention also includes an implant for use in a corneal pocket assay produced by the methods of the present invention. The present invention further includes a plate for forming an implant for a corneal pocket assay, wherein the plate comprises a plurality of substantially uniform perforations. In preferred embodiments, the plate can be sterilized and is composed of metal, preferably stainless steel. The plate is preferably about 0.2 mm in thickness and the perforations are preferably round in cross-section and are about 0.5 mm in diameter. Preferably, the perforations are separated by at least about 0.2 mm. In preferred embodiments, the plate contains 100 perforations in a 10×10 grid, and in preferred embodiments, is substantially planar.

The present invention also includes a method for producing a pill, which includes introducing a curable solution which includes a compound having medicinal properties into perforations in a rigid plate; allowing the solution in the perforations to cure to form pills; and removing the pills from the perforations. The pills preferably are substantially uniform in dimension. The plate is as described above; the solution preferably further includes a slow-release agent, preferably hydroxyethylmethacrylate, and a stabilizing agent. The present invention also includes a pill produced by the method of claim 67.

### BRIEF DESCRIPTION OF THE DRAWINGS

FIGS. 1A, 1B, 1C illustrate typical pellets produced by the prior art nylon mesh method.

FIGS. 1D and 1E illustrate typical pellets produced by methods of the present invention.

FIGS. 2A and 2B illustrate preferred plates of the present invention

### DETAILED DESCRIPTION

The methods of producing implants for corneal pocket assays, methods for performing corneal pocket assays, implants, and devices for forming implants for corneal pocket assays as taught by the present invention provide decreased pellet-to-pellet variability. This improvement provides researchers and others with decreased costs of manufacturing the implants and further, with improved reliability of data resulting from the use of improved pellets obtained by the methods of the present invention, allowing for easier interpretation of experimental results.

In a first embodiment, the present invention includes a method for producing an implant for a corneal pocket assay that includes introducing a curable solution into perforations in a rigid plate and allowing the solution in the perforations to cure to form pellets. The pellets are then removed from the perforations.

Implants of the present invention are useful for corneal pocket assays. An exemplary corneal pocket assay is described in the Kenyon et al. article referenced herein and is known in the art, and has been adapted to several laboratory animals such as mice, rats, and rabbits. The procedure will be briefly discussed in regards to mice. Mice can be anesthetized with about 6.5 mg/ml sodium pentobarbitol. The globes can be proptosed with a Roboz forceps (#RS-5211). Using an operating microscope, a central, intrastromal linear keratotomy (approximately 0.6 mm length) can be performed with a surgical blade (Bard-Parker No. 11, Becton Dickinson, Franklin Lakes, N.Y.) parallel to the insertion of the lateral rectus muscle, and using a modified von Graefe knife (2×30 mm), a lamellar micropocket can be dissected toward the temporal limbus. For implants containing an angiogenic stimulator, the dissection will vary depending on which angiogenic stimulator is used. For bFGF-containing implants, the pocket will be extended to within 0.7 to 1 mm of the temporal limbus and for VEGF-containing pellets, the pocket should be extended to within 0.5 mm of the limbus (due to the relatively weaker angiogenic stimulation of VEGF in this model). A single implant is placed on the corneal surface at the base of the pocket with jeweler's forceps, and using one arm of the forceps, the pellet may be advanced to the temporal end of the pocket. Antibiotic ointment may be applied to the operated eye.

Introduction of a curable solution into perforations in a rigid plate may be performed in any manner known in the art. Any known method of inserting a solution into perforations, openings, or containers and of ensuring an even distribution of solution among the perforations or holes may be used. A preferred method of introduction is to apply a liquid solution with a micropipettor over the area of the perforations or holes in the plate, and a preferred method of distributing the solution among the holes or perforations is to use a cell lifter.

The solution in this step is preferably a liquid solution or suspension, but the solution or suspension may be in any form that allows for manipulation and transfer of the solution. Accordingly, the solution may be in a semi-solid state, a gel-like state, or any other such state. Preferably, the solution is in a liquid state that allows for convenient transfer. The term “solution” as used herein can refer to true solutions or to suspensions.

The method further includes allowing the solution in the perforations to cure to form pellets. Accordingly, solutions of the present invention are curable. As used herein, “curable” refers to the characteristic of becoming less liquid and more solid. For example, solutions can include monomers that polymerize in situ, upon some chemical or physical initiation. Alternatively, solutions can become more solid as water is removed by drying. Preferably, the method of curing is by allowing the solution to dry. A preferred drying time is between about 10 seconds and about 10 minutes, between about 30 seconds and about 7 minutes, between about 1 minute and about 5 minutes, between about 1 minute and about 3 minutes, and preferably between about 2 minutes to about 3 minutes.

This embodiment of the present invention also includes the step of removing the pellets from the perforations. Removal may be accomplished by any method known in the art for removal of formed items from a mold or perforation. Such methods include vacuum removal, removal by shock, removal with tools such as blunt needles or forceps, among others. Preferably, the pellets are removed by dislodging or pushing individual pellets through the individual perforations with a suitable device. A preferred device for pushing individual pellets through individual perforations is a blunted needle. Alternatively, multiple individual pellets can be pushed through multiple individual perforations by using a comb-like or brush-like device.

Preferably, pellets produced by methods of this invention are substantially uniform in dimension. For example, 90% of pellets produced in a batch have less than a 10% to 5% variation in a physical dimension, such as diameter, depth or weight, from the pellet specification. Preferably, at least about 70% of the pellets formed by methods of the present invention are suitable for use in a corneal pocket assay, at least about 75% of the pellets formed by methods of the present invention are suitable for use in a corneal pocket assay, at least about 80% of the pellets formed by methods of the present invention are suitable for use in a corneal pocket assay, and at least about 85% of the pellets formed by methods of the present invention are suitable for use in a corneal pocket assay. More preferably at least about 90% of the pellets are suitable for use in a corneal pocket assay, and most preferably at least about 95% of the pellets are suitable for use in a corneal pocket assay. Suitable pellets for use in a corneal pocket assay are solid white. Opaque or cracked pellets are not suitable.

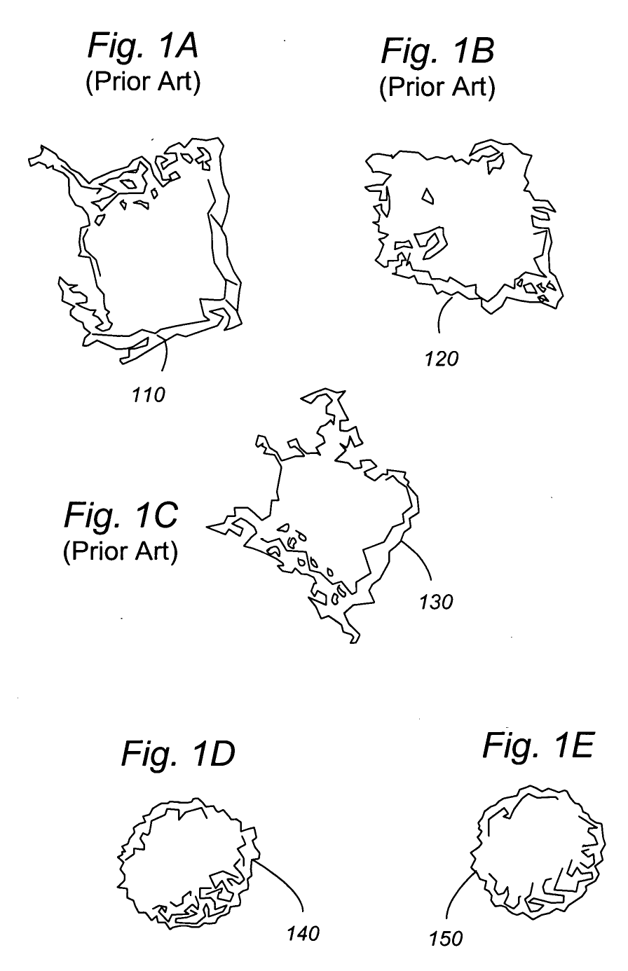

FIGS. 1A, 1B, and 1C show typical pellets produced by the prior art nylon mesh method 110, 120, and 130. FIGS. 1D and 1E show typical pellets produced by methods of the present invention 140 and 150. In pellets 110, 120, and 130, the pellets vary in dimension from one to another due to distortion inherent in a flexible nylon mesh and the method of production. Additionally, the pellets 110, 120 and 130 have an extension or an overhang, i.e., additional dried solution extending out from the main body of the pellet due to the use of nylon mesh. This additional material contributes significantly to pellet variability in both dimension and in volume or amount per pellet. In contrast, pellets 140 and 150 are substantially identical in dimension to each other, and lack any overhang or extension.

In this embodiment, a rigid plate is used to form the pellets. Preferably, a plate is a smooth, substantially flat body of substantially uniform thickness. The thickness of the plate is selected such that the resulting pellet will have a desired thickness. The plate is planar, relatively stiff and not flexible or pliant to a significant degree. The plate is also preferably capable of being readily sterilized. The plate is compatible with aqueous solutions so that when a solution of the present invention is applied, it disperses well with a uniform spread on the plate at a rapid rate. In this manner, the amount of liquid volume necessary to be contacted with perforations is reduced, thereby minimizing costs. Materials suitable for use for plates include stainless steel or plastic. A particularly preferred plate material is stainless steel.

Perforations in the plate are formed to match the desired dimensions of the pellet. Perforations may be introduced into a plate by any method known in the art. Perforations are preferably round and through the entire thickness of the plate so that a resulting pellet will be cylindrical. The perforations are preferably, at a minimum, rounded, such as oval-shaped, without any sharp corners such as in a square or rectangle. Dimensions of the pellet will vary in accordance with the particular laboratory animal model being used. Where the animal model being used is a mouse model, a preferred plate thickness is about 0.2 mm. Preferably, the perforations will be round in cross-section and about 0.5 mm in diameter. The perforations can be separated from each other on the plate. Preferably, the perforations are placed close to one another as to minimize any wastage of solution as it is spread around the plate to fill the perforations. A preferred distance for separation of perforations is about 0.5 mm to 2 mm.

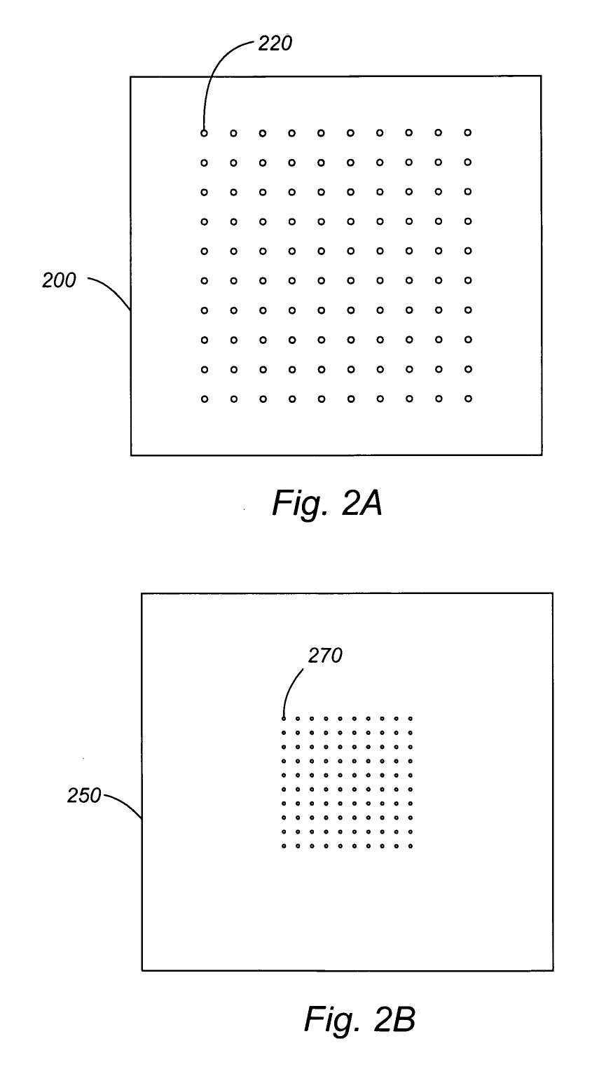

FIGS. 2A and 2B demonstrate preferred plates of the present invention. In FIG. 2A, plate 200 has multiple perforations 220, (one hundred perforations arranged in a 10×10 grid), where each perforation is 0.5 mm in diameter and 0.2 mm in depth, and each perforation is separated from each other by 0.5 mm. The perforations extend all the way through the plate 200. In FIG. 2B, plate 250 has multiple perforations 270, (one hundred perforations arranged in a 10×10 grid), each perforation is 0.5 mm in diameter and 0.2 mm in depth, and each perforation is separated from each other by 2 mm. The perforations extend all the way through the plate 250.

The solution preferably contains a biologically active agent which acts to induce the desired disease or disorder in the animal model. Such an animal model may be classified as an induced (experimental) disease model. As the name implies, induced models are healthy animals in which the condition to be investigated is experimentally induced. Most induced models are partial or isomorphic because the etiology of a disease experimentally induced in an animal is often different from that of the corresponding disease in the human. Few induced models completely mimic the etiology, course, and pathology of the target disease in the human. Other types of animal models suitable for the present invention include spontaneous (genetic) disease models, transgenic disease models, negative disease models (i.e., species where certain diseases do not develop), and orphan disease models. The biologically active agent may include any agent which induces the desired disease or disorder. Particularly relevant to the present invention are biologically active agents which induce angiogenesis and/or neovascularization, particularly in the cornea. It is particularly preferable for the agent to directly stimulate blood vessel formation rather than indirectly stimulate blood vessel formation by inducing inflammation. Biologically active agents which stimulate angiogenesis and/or neovascularization include, for example, basic fibroblast growth factor (bFGF), vascular endothelial growth factor (VEGF), endotoxin, tumor cells, interleukin 2, and injury. Particularly preferred biologically active agents include bFGF and VEGF. Most preferred is bFGF. An amount of biologically active agent to use in the solution is the amount which will induce the desired disease or disorder in the animal model reproducibly and to a significant degree. Where the biologically active agent is bFGF and the laboratory animal is a mouse, the preferred amount per pellet is between about 60 ng and about 120 ng, between about 70 ng and about 110 ng, between about 80 ng and about 100 ng, and preferably about 90 ng per pellet. Where the biologically active agent is VEGF and the laboratory animal is a mouse, the preferred amount per pellet is between about 150 ng and about 210 ng, between about 160 ng and about 200 ng, between about 170 ng and about 190 ng, and preferably about 180 ng per pellet.

In a preferred embodiment, the solution is formulated to form a pellet in a sustained release formulation. A sustained release formulation increases the length of time that a biologically active agent(s) is available to cause a biologic effect after insertion of a pellet containing a biologically active agent into a corneal pocket. Any compound known in the art which is capable of being formulated with a biologically active agent and which will increase the length of time that the biologically active agent(s) is available is suitable for use in the present invention. Such a compound may be referred to as a slow-release agent. A preferred slow-release agent for increasing the length of time for bioavailability of a biologically active agent is a slow-release polymer. Many slow-release polymers are known in the art, and are suitable for use with this invention. A preferred type of slow-release polymer includes swellable hydrogel systems. Swellable hydrogels include but are not limited to hydroxyethylmethacrylate (HEMA), (also known by the trade name HYDRON®, available from Interferon Sciences, New Brunswick, N.J.) polyethyleneglycolmethacrylate (PEGMA), cellulose ether hydrogels, comprising cross-linked hydroxypropyl cellulose, methyl cellulose, and hydroxypropylmethyl cellulose; calcium-crosslinked alginate; crosslinked polyvinyl alcohols and Poloxamers (Pluronics). A suitable slow-release agent will not induce inflammation in the cornea or induce only minimal inflammation. A preferred slow-release polymer is hydroxyethylmethacrylate. The amount of slow-release agent will vary in accordance with the proportions of the other ingredients and the length of time for sustained release desired. Depending on the length of sustained release desired, the amount of slow-release agent to use will vary from none to about 15% (w/v). In a preferred embodiment, the amount of slow-release agent to use will be in the range of between about 12% w/v and about 13% w/v. A preferred embodiment, the amount of slow-release agent to use is 12% (w/v) VEGF. A preferred embodiment, the amount of slow-release agent to use is 13% (w/v) bFGF.

Preferably, the solution will further comprise a stabilizing agent. A stabilizing agent can help stabilize a biologically active agent and/or other ingredients against degradation, loss of potency and/or loss of biological activity, all of which can occur during formation of the sustained release composition having the biologically active agent dispersed therein, and/or prior to and during in vivo release of the biologically active agent. In one embodiment, stabilization can result in a decrease in the solubility of the biologically active agent, the consequence of which is a reduction in the initial release of biologically active agent, in particular, when release is from a sustained release composition. In addition, the period of release of the biologically active agent can be prolonged. Stabilization of the biologically active agent can be accomplished, for example, by the use of a stabilizing agent or a specific combination of stabilizing agents. The stabilizing agent can be present in the mixture. “Stabilizing agent,” as that term is used herein, is any agent which binds or interacts in a covalent or non-covalent manner or is included with the biologically active agent. A stabilizing agents suitable for use in the invention includes sucralfate (sucrose aluminum hydroxide). A preferred stabilizing agent is sucralfate. A suitable stabilizing agent will not induce inflammation in the cornea or induce only minimal inflammation. The amount of stabilizing agent will vary in accordance with the proportions of the other ingredients and the necessity for stabilization. Depending the degree of stabilization desired, the amount of stabilizing agent to use will vary from none to about 30% (w/v). In a preferred embodiment, the amount of stabilizing agent will in the range of between about 20% (w/v) and about 25% (w/v).

The solution may optionally further include an excipient. Such excipients can be added to the compositions of the present invention such as, for example, to maintain the potency of the various components over the duration of release and to modify polymer degradation. One or more excipients can be added to the solution. Suitable excipients include, for example, acidic or basic agents, carbohydrates, amino acids, fatty acids, surfactants, and bulking agents. Such excipients are known to those of ordinary skill in the art and the amount necessary or desirable can be determined by such skilled workers.

Another embodiment of the present invention includes a method for producing an implant for a corneal pocket assay. This method includes introducing a curable solution into perforations in a perforated member and allowing the solution in the perforations to form pellets. The pellets are removed from the individual perforations by pushing individual pellets through individual perforations. A perforated member can be, for example, a rigid plate having perforations, as described above, or other similar structure suitable for forming similar pellets.

In yet another embodiment, the present invention includes a method for performing a corneal pocket assay for testing a putative pharmaceutically active agent in laboratory animals. The method includes placing a pellet into a corneal pocket of each of at least two laboratory animals, wherein the pellets are substantially uniform in dimension. The putative pharmaceutically active agent is introduced into at least one of the laboratory animals, and the pharmacologic activity of the agent is evaluated.

In this embodiment, the pharmaceutically active agent is introduced into at least one of the laboratory animals. The term “pharmaceutically active agent,” as used herein, is an agent which, when released in vivo, possesses the desired biological activity, for example, therapeutic, diagnostic and/or prophylactic properties in vivo. It is understood that the term includes stabilized- and or extended release-formulated pharmaceutically active agents. The terms “pharmaceutically active agent,” “therapeutic, prophylactic or diagnostic agent,” “drug,” “active agent,” and “agent” are used interchangeably herein.

Examples of suitable pharmaceutically active agents include, but are not limited to, antiangiogenic agents; antipsychotic agents; antitumor agents; antibiotics; antipyretic, analgesic and anti-inflammatory agents; antitussives and expectorants; sedatives; muscle relaxants; antiepileptics; antiulcer agents; antidepressants; antiallergic agents; cardiotonics; antiarrhythmic agents; vasodilators; hypotensive diuretics; antidiuretic agents; anticoagulants; hemostatic agents; antituberculous agents; hormones; and narcotic antagonists. Additional pharmaceutically active agents suitable for use in the invention include, but are not limited to, proteins, muteins and active fragments thereof, such as immunoglobulins, antibodies, cytokines (e.g., lymphokines, monokines, chemokines), interleukins, interferons (β-IFN, α-IFN and γ-IFN), erythropoietin, nucleases, tumor necrosis factor, colony stimulating factors, insulin, enzymes (e.g., superoxide dismutase, tissue plasminogen activator), tumor suppressors, blood proteins, hormones and hormone analogs (e.g., growth hormone, adrenocorticotropic hormone and luteinizing hormone releasing hormone (LHRH)), vaccines (e.g., tumoral, bacterial and viral antigens), antigens, blood coagulation factors; growth factors; peptides such as protein inhibitors, protein antagonists, and protein agonists; nucleic acids, such as antisense molecules; oligonucleotides; and ribozymes. A preferred pharmaceutically active agent is an angiogenesis inhibitor (a pharmaceutically active agent which acts to inhibit angiogenesis.) The compounds screened in an assay referenced in the present invention may or may not have any pharmaceutical activity, hence they are properly described as having putative pharmaceutical activity and as putative pharmaceutically active agents.

An appropriate amount of the putative pharmaceutically active agent to add may be determined by one of skill in the art. Generally, the pharmaceutically active agent is formulated in an isotonic aqueous solution and may be solubilized by any method known in the art. The pharmaceutical formulation may also contain excipients known in the art. The solutions may contain from about 0.01% (w/w) to about 90% (w/w) of the pharmaceutically active agent (dry weight of composition). The amount of agent can vary depending upon the desired effect of the agent, the planned release levels, and the time span over which the agent is to be released.

The pharmaceutically active agent may be introduced into or administered to the laboratory animal by any method known in the art. Generally, pharmaceutically active agents may be introduced intravenously, intraperitoneally, intradermally, intraocularly, topically or subcutaneously. Agents may be dosed continuously or intermittently as determined by factors by one known in the art, including such factors as design of the experimental protocol, convenience, half life of the agent, and so on.

Pharmaceutical activity of the agent may be evaluated by methods known in the art. For example, eyes may be examined by slit lamp biomicroscopy (Nikon FS-2, Tokyo, Japan) on successive postoperative days after pellet implantation. Animals are preferably anesthetized with sodium pentobarbitol, the eyes were proptosed, and the maximum vessel length of the neovascularization zone, extending from the base of the limbal vascular plexus toward the pellet, may be measured with a linear reticule through the slit lamp. The contiguous circumferential zone of the neovascularization may be measured. A preferred laboratory animal is a mouse. Other preferred laboratory animals include rabbits and rats. Preferably the laboratory animals used are of a known strain and animals used in an individual experiment are of the same strain.

The present invention also includes pellets that are produced by the methods of the present invention. The present invention further includes implants for use in a corneal pocket assay produced by the methods of the present invention.

In a further embodiment, the present invention also includes a plate for forming an implant for a corneal pocket assay, wherein the plate comprises a plurality of substantially uniform perforations. For example, a substantially uniform perforation is one having less than a 10% to 1% variation in a physical dimension such as diameter or depth of the perforation from the average of the perforations on the plate. Preferably, the plate can be sterilized by methods known in the art. Such methods include high temperature sterilization such as autoclaving, liquid immersion methods using chemical sterilants such as hydrogen peroxide, ultraviolet irradiation, exposure to ionizing radiation, and the like. A preferred method of sterilizing is by autoclaving.

A further embodiment of the present invention is a process for making pills and pills produced thereby. The process includes introducing a curable solution into perforations in a rigid plate and allowing the solution in the perforations to cure to form pills. The pills are then removed from the perforations. The method is similar to the process described herein for producing a corneal pocket implant, except that the curable solution includes a compound having some medicinal properties. For example, such a compound can be a type of pharmaceutically active agent as described above in relation to putative pharmaceutically active agents in the corneal pocket implant assay. Further, components of the curable solution for making the pills must be safe for ingestion by the patient (whether human or other animal) to whom the pill will be administered. This process provides significant advantages in the manufacture of pills, including the ability to rapidly produce pills of uniform size and composition.

The present invention, while disclosed in terms of specific methods, products, and organisms, is intended to include all such methods, products, and organisms obtainable and useful according to the teachings disclosed herein, including all such substitutions, modifications, and optimizations as would be available to those of ordinary skill in the art. The following examples and test results are provided for the purposes of illustration and are not intended to limit the scope of the invention.

### EXAMPLES

### Example 1

This Example demonstrates the preparation of pellets or implants using a stainless steel plate.

Ten microgram (μg) bFGF or 20 μg VEGF was dissolved in 8 microliters (μl) saline solution. 3-4 milligram (mg) carafate (sucralfate) was added and mixed to a uniform suspension. To the suspension, 8 μl of a 12% (w/v). Hydron solution (obtained from Hydro Med Sciences) was added to the suspension for VEGF, and 8 μl of a 15% (w/v) Hydron solution was added to the suspension bFGF, and mixed to a uniform suspension. The solution was pipetted with a micropipettor onto a stainless steel plate as depicted in FIG. 2A, over the plate surface, over the area of the perforations or holes. A cell lifter was used to spread the material over the holes. The solution was then allowed to dry for one to two minutes, and the pellets were then pushed or popped out of the holes or perforations using a blunted, single-point tattoo needle. Yield was 90-100 pellets, with 90-95 suitable for implant. The actual yield was 92 acceptable pellets.

### Example 2

This Example demonstrates a corneal pocket assay with the angiogenesis stimulator VEGF in accordance with the present invention.

Pellets were made either with or without VEGF (200 μg per pellet) in the manner as described in Example 1. Four treatment regimens were followed: implantation with +VEGF pellet followed by vehicle treatment; implantation with null VEGF pellet followed by vehicle treatment; implantation with +VEGF pellet, with an anti-angiogenesis agent (GL1) at either 25 or 12.5 mg/kg, ip, bid X 7.

Female mice of strain C57BL/6, 5-6 weeks of age, were anesthetized with methoxyflurane and the eyes topically anesthetized with 0.5% proparacaine (Opththetic, Alcon, Tex.). The globes were proptosed with Roboz forceps, model RS-5211. Using an operating microscope, a central, intrastromal linear keratotomy (approximately 0.6 mm length) was performed with a surgical blade (Bard-Parker No. 11, Becton Dickinson, Franklin Lakes, N.Y.) parallel to the insertion of the lateral rectus muscle, and a modified von Graefe knife (2×30 mm) was used to dissect a lamellar micropocket toward the temporal limbus. For VEGF-containing pellets, the pocket was extended to within 0.5 mm of the limbus (due to the relatively weaker angiogenic stimulation of VEGF in this model). A single implant was placed on the corneal surface at the base of the pocket with jeweler's forceps, and using one arm of the forceps, the pellet was advanced to the temporal end of the pocket. Antibiotic ointment was applied once to the operated eye.

Eyes were routinely examined by slit lamp biomicroscopy (Nikon FS-2, Tokyo, Japan) on postoperative days 1 through 6 after pellet implantation. Mice were anesthetized with avertin, the eyes were proptosed, and the maximum vessel length of the neovascularization zone, extending from the base of the limbal vascular plexus toward the pellet, was measured with a linear reticule through the slit lamp. The contiguous circumferential zone of the neovascularization was measured as 1.7 mm² on day 8. Results of the study are shown in Table 1.

TABLE 1
Neovascularization in corneal pocket assay in response to VEGF as
expressed as length of vascular extension in mm compared to response with angi-
angiogenesis agent (GL1).

| | | | | | | area of | |
|---|---|---|---|---|---|---|---|
| | | | μg/pellet | | mg/kg | neovascularization in | angiogenesis % of |
| Group | n | Factor | factor | Agent | agent | mm Mean ± SEM(n) | positive control |
| 1 | 6 | VEGF− | 0 | none | 0 | 0.0 ± 0.0 (6) | 0% |
| 2 | 8 | VEGF+ | 200 | none | 0 | 1.7 ± 0.2 (8) | 100% |
| 3 | 8 | VEGF+ | 200 | GL1 | 25 | 1.0 ± 0.1 (8) | 59% |
| 4 | 8 | VEGF+ | 200 | GL1 | 12.5 | 1.2 ± 0.2 (8) | 71% |

Results of this study show the successful use of pellets of the present invention in a corneal pocket assay demonstrating the anti-angiogenesis effects of known angiogenesis inhibitors. Use of pellets from the stainless steel plate had increased uniformity and shape thereby making implant easier and more efficient.

The principles, preferred embodiments and modes of operation of the present invention have been described in the foregoing specification. The invention which is intended to be protected herein should not, however, be construed as limited to the particular forms disclosed, as these are to be regarded as illustrative rather than restrictive. Variations and changes may be made by those skilled in the art without departing from the spirit of the present invention. Accordingly, the foregoing best mode of carrying out the invention should be considered exemplary in nature and not as limiting to the scope and spirit of the invention as set forth in the appended claims.

1. A method for producing an implant for a corneal pocket assay, comprising:
introducing a curable solution into perforations in a rigid plate;
allowing the solution in the perforations to cure to form pellets; and
removing the pellets from the perforations.

2. The method of claim 1, wherein pellets produced by the method are substantially uniform in dimension.

3. The method of claim 1, wherein at least about 70% of the pellets are suitable for use in the corneal pocket assay.

4. (canceled)

5. (canceled)

6. The method of claim 1, wherein at least about 95% of the pellets are suitable for use in the corneal pocket assay.

7. The method of claim 1, wherein the plate is metal.

8. (canceled)

9. The method of claim 1, wherein the plate is about 0.2 mm in thickness.

10. The method of claim 1, wherein the perforations are round in cross-section.

11. The method of claim 10, wherein the perforations are about 0.5 mm in diameter.

12. (canceled)

13. (canceled)

14. The method of claim 1, wherein the solution comprises a growth factor.

15. The method of claim 14, wherein the growth factor is selected from the group consisting of bFGF and VEGF.

16. The method of claim 1, wherein the solution comprises a slow-release agent.

17. (canceled)

18. The method of claim 1, wherein the solution comprises a stabilizing agent.

19. (canceled)

20. The method of claim 1, wherein the solution comprises sucralfate, hydroxyethylmethacrylate, and a growth factor.

21. (canceled)

22. A method for producing an implant for a corneal pocket assay, comprising:
introducing a solution into perforations in a perforated member;
allowing the solution in the perforations to form pellets;
removing pellets from the perforated member by pushing individual pellets through individual perforations.

23-42. (canceled)

43. A method of performing a corneal pocket assay for testing a putative pharmaceutically active agent in laboratory animals, comprising:
a) placing a pellet into a corneal pocket of each of at least two laboratory animals, wherein the pellets are substantially uniform in dimension;
b) introducing the pharmaceutically active agent into at least one of the at least two laboratory animals; and
c) evaluating the pharmaceutical activity of the agent.

44. The method of claim 43, wherein the putative pharmaceutically active agent comprises a putative anti-angiogenesis agent.

45. The method of claim 43, wherein the pellets comprise a growth factor.

46. The method of claim 45, wherein the growth factor is selected from the group consisting of bFGF and VEGF.

47. The method of claim 43, wherein the pellets comprise a slow-release agent.

48. (canceled)

49. The method of claim 43, wherein the pellets comprise a stabilizing agent.

50. (canceled)

51. The method of claim 43, wherein the pellets comprise sucralfate, hydroxyethylmethacrylate, and a growth factor.

52. The method of claim 43, wherein the laboratory animal is a mouse.

53. (canceled)

54. (canceled)

55. (canceled)

56. (canceled)

57. A plate for forming an implant for a corneal pocket assay, wherein the plate comprises a plurality of substantially uniform perforations.

58. The plate of claim 57, wherein the plate can be sterilized.

59. The plate of claim 57, wherein the plate is metal.

60. (canceled)

61. The plate of claim 57, wherein the plate is about 0.2 mm in thickness.

62. The plate of claim 57, wherein the perforations are round in cross-section.

63. The plate of claim 62, wherein the perforations are about 0.5 mm in diameter.

64. (canceled)

65. (canceled)

66. The plate of claim 57, wherein the plate is substantially planar.

67-73. (canceled)
